(19) United States
(12) Patent Application Publication
(10) Pub. No.: US 2003/0149248 A1
(43) Pub. Date: Aug. 7, 2003

(54) Method to evaluate the systemic exposure in toxicological and pharmacological studies

(76) Inventors: Riccardo Bellazzi, Pavia (IT); Giuseppe De Nicolao, Milan (IT); Paolo Magni, Pavia (IT); Italo Poggesi, Varese (IT); Maurizio Rocchetti, Milan (IT)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/239,201

(22) PCT Filed: Mar. 22, 2001
(86) PCT No.: PCT/EP01/03252

(30) Foreign Application Priority Data
Mar. 30, 2000 (GB) 0007788.3

(51) Int. Cl. C07K 14/00

(52) U.S. Cl. 530/405

(57) ABSTRACT

A method of estimating the average exposure within a population of subjects to a pharmacological substance, administered according to a given protocol, by estimating the area under the concentration curve (AUC), characterised in that the area under the population concentration curve is estimated by the steps of obtaining measurements of the drug concentrations in each of the subjects at any time during the study, independently from the time when measurements of the drug concentration level in the other subjects are being taken; building a hierarchical stochastic model composed of the population and of the individual levels; determining the posterior probability distribution of the average AUC from the sample data and hence the average population AUC. The exposure of an individual and its precision within the sample of individuals can be determined by determining the posterior probability distribution of the individual AUC from the sample data. The posterior probability model may be obtained by using a Markov Chain Monte Carlo algorithm, such as Gibbs Sampling.

The present invention relates to pharmacokinetic studies which are used to evaluate the systemic exposure of animals or subjects to a pharmacological substance. It also relates to the correlation of the descriptor of such exposure to therapeutic and toxic effects (pharmacodynamics) of the drug.

One of the most important measures of the exposure of animals or subjects to a pharmacological substance is the area under the plasma concentration-time curve (AUC). The area under the curve is the integral of the concentration of the drug in plasma with respect to time. In practice it is rarely possible to take continuous measurements of the plasma concentration of the drug and instead samples are collected from the subjects at times distributed over the exposure time. In cases were it is possible to collect a sufficient number of samples from the subject the AUC and hence the exposure of the drug can easily be determined by numerical integration techniques such as the linear trapezoidal rule. In the following text, the term AUC will indicate the area under the plasma concentration-time curve calculated up to some fixed time (AUC0-t). However, all the procedures can be extended to the total exposure, i.e., the AUC calculated up to infinite time, by means of a suitable mathematical model describing the decline of the plasma concentration-time curve.

Furthermore, because population values, such as population AUC and population mean, are obtained via sample values, that which is estimated is a sample AUC and sample mean, respectively, which, in turn, are unbiased estimates of the population AUC and population mean. In the following text the terms population AUC and population mean will be used for simplicity.

There are two aspects of pharmacokinetic and pharmacokinetic/pharmacodynamic studies. From one side, the aim is to determine the exposure of a specific individual to the drug to specify the dosing regimen required in that individual to achieve a specific therapeutic goal. From the other side, the aim is to determine the average exposure to the drug that would be experienced by the individuals in a population. One method of deriving the average exposure to the drug is to determine the AUC for several individuals and then use conventional descriptive statistics to have an estimate of the population AUC. Alternatively, the measure of the population AUC can be determined directly from all the individual measurements of plasma concentrations considered together.

An important further consideration in pharmacodynamic studies is the practical limitations of the sampling protocols. In many cases the number of samples that can be collected from an individual will be limited by technical, ethical and cost reasons. For example in toxicokinetic studies only a few samples can be taken in order not to perturb the subjects under observation. Furthermore in experiments which involve destructive measurements (in which the subject is sacrificed) only one sample can be collected from each subject. An example of such a situation is the study of metabolite levels in an internal organ. In these cases the only possible option is to consider all of the individuals' samples together.

Solutions have been proposed to determine the central tendency (e.g. mean) and the dispersion (e.g. SE) of the AUC of the population directly from the individuals' samples. For example the Bailer method (“Testing for the Equality of Area Under the Curves When Using Destructive Measurement Techniques”, Journal of Pharmacokinetics and Biopharmaceutics, Vol 16, No. 3, 1988), which can be used to estimate the descriptive statistics of the AUC when destructive sampling is required and the Yeh method (“Estimation and Significant Tests of Area Under the Curve Derived from Incomplete Blood Sampling”, Proceedings of the American Statistical Association, 1990, p. 7481) which can be used to estimate the average AUC and the corresponding SE when the population is subdivided in different groups and a few samples are collected from each subject at different times.

Considering a study with the following parameters:

N subjects are utilized in the study,

samples are taken at m time points (t₁, . . . , tₘ),

at each sampling time nⱼ subjects are randomly selected to form m sampling groups,

such that

$${\sum }_{j = 1}^{m} {n}_{j} = N ,$$

, ie. every subject is part of one of the sampling groups, and

the measurement taken in the i-th subject at the time tⱼ is denoted yᵢⱼ.

Then the Bailer method calculates the estimate of the population AUC and its standard error (SE_{AUC}) as:

$$\begin{matrix}AUC = {\sum }_{j = 1}^{m} {a}_{j} {c}_{j} & ( 1 ) \\ {SE}_{AUC} = \sqrt{{\sum }_{j = 1}^{m} {a}_{j}^{2} {s}_{j}^{2} / {n}_{j}} \\ where \text{:} & ( 2 ) \\ {c}_{j} = {\sum }_{i = 1}^{{n}_{j}} \frac{{y}_{ij}}{{n}_{j}} & ( 3 ) \\ {s}_{j}^{2} = {\sum }_{i = 1}^{{n}_{j}} \frac{{( {y}_{ij} - {c}_{j} )}^{2}}{( {n}_{j} - 1 )} & ( 4 ) \\ {a}_{j} = \{ \begin{matrix}( {t}_{2} - {t}_{1} ) / 2 & for j = 1 \\ ( {t}_{j + 1} - {t}_{j - 1} ) / 2 & for j > 1 and j < m \\ ( {t}_{m} - {t}_{m - 1} ) / 2 & for j = m \end{matrix} & ( 5 )\end{matrix}$$

In order to find the standard error, an estimate, within each sampling group, of the average variation sⱼ of the individual measurements yᵢⱼ from the average of the group cⱼ is calculated according to (4). The standard error SE_{AUC} is then calculated according to (2) by applying the estimate of the average variation sⱼ over the width of each section.

One problem with the Bailer method is that in order to calculate the standard error it is assumed that the measured points of the concentration curve are independent of each other. Furthermore the Bailer method makes no distinction between measurement error and inter-individual variability.

The Yeh method is a slight generalization of the Bailer method. Consider a further study in which the parameters of the study are:

N subjects are utilized in the study,

the subjects are divided randomly into S groups (hereafter called sessions),

samples are taken at m time points,

each session contains nₖ subjects,

such that

$${\sum }_{k = 1}^{S} {n}_{k} = N$$

ie. each subject is allocated to one session,

each of the nₖ subjects in each session is sampled more than once,

each of the nₖ subjects in each session is sampled at mₖ time points,

the sampling times of the sessions are different, and

the measurement taken in the i-th subject of the group k at time j is yᵢⱼₖ.

Then the Yeh method calculates the estimate of the population AUC and its standard error (SE_{AUC}) as:

$$\begin{matrix}AUC = {\sum }_{k = 1}^{S} {f}_{k} & ( 6 ) \\ {SE}_{AUC} = \sqrt{{\sum }_{k = 1}^{S} {s}_{k}^{2} / {n}_{k}} \\ where \text{:} & ( 7 ) \\ {f}_{k} = \frac{1}{{n}_{k}} {\sum }_{i = 1}^{{n}_{k}} {f}_{ik} & ( 8 ) \\ {f}_{ik} = {\sum }_{j ε {m}_{k}} {a}_{j} {y}_{ijk} & ( 9 ) \\ {S}_{k}^{2} = {\sum }_{i = 1}^{{n}_{k}} \frac{{( {f}_{ik} - {f}_{k} )}^{2}}{{n}_{k} - 1} & ( 10 )\end{matrix}$$

The conditions for aⱼ are the same as for the Bailer method as shown in (5). Equation (9) shows that the first step is to calculate the estimate of the AUC for each individual in each session by using a method similar to that which was used in the Bailer method to calculate the estimate of the population AUC from the average concentrations at each of the sampling times. An estimate for the average AUC for each session is then calculated according to (8) and finally the estimate for the population AUC is calculated according to (6) by summing the contributions of each of the sessions.

The standard error is again obtained by assuming that the sampled points on the concentration curve related to different sessions are independent of each other. Also the Yeh method, as in the Bailer method, does not make a distinction between measurement error and inter-individual variability. Moreover the sampling protocol is restrictive as all the subjects in each session are sampled at the same time points and the subjects of different sessions are sampled at different time points.

In addition, with these methods it is only possible to calculate descriptive statistics (e.g., mean, SE) of the exposure to the drug in a sample of individuals. This means that it is not possible to calculate the individual values of the exposure to the drug, which, in turn, does not allow one to apply the pharmacokinetic/pharmacodynamic relationships to identify the individual dosing regimen able to realize the optimal therapeutic outcome.

It is therefore the aim of the present invention to provide an improved method of deducing the average exposure to a drug with fewer limitations on the sampling pattern and which can more accurately assess the standard error, taking account of the effects of inter-individual variability and measurement error. It is a further aim of the invention to provide a method which evaluates the individuals' exposure to a drug.

According to a first aspect of the present invention there is provided a method of determining the average exposure within a population of subjects to a pharmacological substance administered according to a given protocol by determining the area under the concentration curve, characterised in that the area under the population concentration curve is estimated by the steps of:

obtaining measurements of the drug concentrations in each of the subjects at any time during the study, independently from the time when measurements of the drug concentration level in the other subjects are being taken;

building a hierarchical stochastic model composed of the population and of the individual levels;

determining the posterior probability distribution of the average AUC from the sample data and hence the average population AUC.

According to a further aspect of the present invention there is provided a method of determining the exposure of an individual and its precision within the sample of individuals given a pharmacological substance administered according to a given protocol by determining the area under the concentration-time curve, characterised in that the area under the individual concentration-time curve is estimated by the steps of:

obtaining measurements of the drug concentrations in each of the subjects at any time during the study, independently from the time when measurements of the drug concentration level in the other subjects are being taken;

building a hierarchical stochastic model composed of the population and of the individual levels;

determining the posterior probability distribution of the individual AUC from the sample data.

According to a further aspect of the present invention there is provided a computer program comprised of computer code means for estimating the average total exposure within a population of subjects to a pharmacological substance, administered according to a given protocol, by estimating the area under the concentration curve (AUC); the computer code means being comprised of means to:

record measurements of the drug concentration level in each of the subjects at any time within the study, independently from the time when measurements of the drug concentration level in the other subjects are being taken;

determine the posterior probability distribution of the population AUC from the recorded sample data using a hierarchical stochastic model of the population; and

hence determine the average population AUC.

According to a further aspect of the present invention there is provided a computer program comprised of computer code means for determining the exposure of an individual and its precision within the sample of individuals given a pharmacological substance administered according to a given protocol by determining the area under the concentration-time curve, the computer code means being comprised of means to:

record measurements of the drug concentrations in each of the subjects at any time during the study, independently from the time when measurements of the drug concentration level in the other subjects are being taken;

build a hierarchical stochastic model composed of the population and of the individual levels; and

determine the posterior probability distribution of the individual AUC from the sample data.

The method of the present invention is advantageous because it can be applied to any arbitrary sampling scheme. Furthermore it allows the rigorous derivation of the posterior distribution of each parameter which means that the precision of the estimates can be made as well as the point estimates. This method is also advantageous because it provides estimates of the intra- and inter-subject variability.

The invention will now be described by way of a non-limiting example with reference to the drawing, in which:

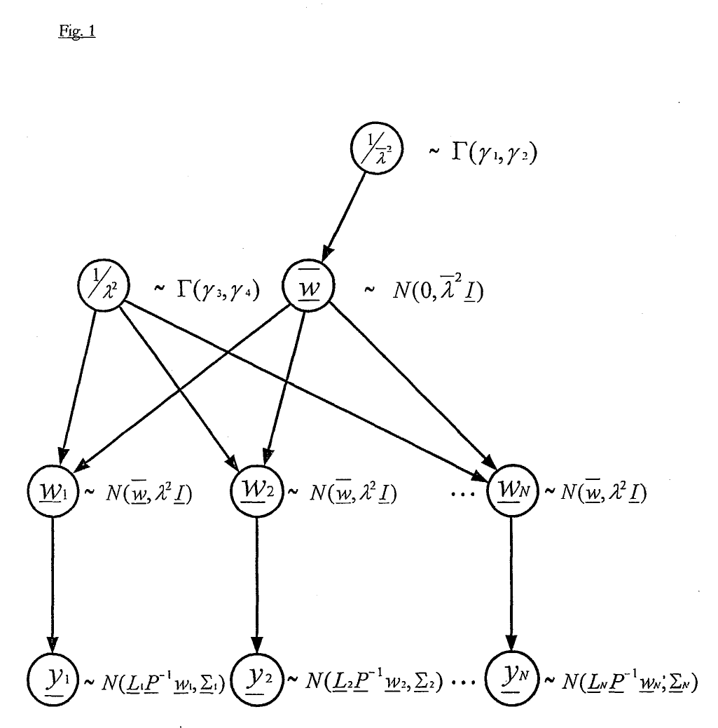

FIG. 1 is a representation of the stochastic population model according to the invention;

The present invention is based upon a stochastic model of both the individual concentration curves and the population curve. A hierarchical model is used to describe the population model, whereas random walks describe the individual plasma concentration curves and the population curve. Model estimation is performed according to a Bayesian approach based on a Markov Chain Monte Carlo stochastic simulation algorithm. In a preferred embodiment, described below, Gibbs Sampling is used to derive the population AUC, the individuals' AUC and the other parameters describing the inter-individual variability.

In a study carried out according to the present invention there are no restrictions to the sampling protocol. The parameters of the study are therefore:

N subjects are utilized in the study,

nᵢ measurements are taken for each subject,

the set T {t₁, . . . , tₘ} represents all of the times when a sample is taken from any subject,

the measurement of the i-th subject at time tⱼ is yᵢⱼ,

By deriving the posterior probability distribution of the population concentration curve \overline{x}ⱼ (j=1, . . . , m) it will be possible, by for example the trapezoidal rule, to compute the probability distribution of the population AUC. A posterior probability distribution is the probability distribution that a parameter lies in each of the various regions of the set of all its possible values after (posterior to) the observation of any values from a distribution which depends on the parameter. Thus in this case the posterior distribution will be the distribution of the probability that the concentration will be at a given level at a certain time.

In the present invention it is assumed that the curve \overline{x}ⱼ is, to a certain degree, regular. This is a reasonable assumption given that the signal that the invention relates to is a biological signal. A simple stochastic model of a regular signal is given by a random walk and therefore \overline{x}ⱼ is modelled by:

\overline{x}ⱼ=\overline{x}ⱼ−1+\overline{ω}ⱼ,\overline{x}₀=0 (11)

where the variables \overline{ω}ⱼ are independent and normally distributed with zero mean and variance \overline{λ}². The sampling times t₁, . . . tₘ are assumed to have been chosen properly to ensure that when the signal is less regular the sampling is more frequent. Equation (11) is usefully be rewritten in the form:

P\overline{x}=\overline{w} (12)

where:

\overline{x}=[\overline{x}₁, . . . ,\overline{x}ₘ]^{T} (13)

\overline{w}=[\overline{w}₁, . . . ,\overline{w}ₘ]^{T} (14)

$$\underline{P} = [ \begin{matrix}1 & 0 & … & 0 \\ - 1 & 1 & … & 0 \\ ⋮ & ⋰ & ⋰ & ⋮ \\ 0 & ⋯ & - 1 & 1\end{matrix} ] \qquad ( 15 )$$

\overline{w}^{˜}.N(0,\overline{λ}²I) (16)

I is the identity matrix

A similar modelization is also made for the individual concentration curves (xᵢⱼ), assuming that the assumption of regularity made above is also true at the individual level. Thus for the i-th subject:

xᵢⱼ=xᵢⱼ−1+wᵢⱼ,xᵢ₀=0 (17)

where the variables wᵢⱼ are independent and normally distributed with mean \overline{w}ⱼ and variance λ². In other words, the individual curves are random walks with expected value equal to the population curve and inter-individual variability modelled by λ². This too is usefully rewritten as:

Pxᵢ=wᵢ (18)

where:

xᵢ=[xᵢ₁, . . . ,x_{im]}^{T} (19)

wᵢ=[wᵢ₁, . . . ,wᵢₘ]^{T} (20)

wᵢ^{˜}N(\overline{w},λ²I) (21)

The measurement error is also modelled, assuming that each measurement is affected by an additive error:

yᵢⱼ=xᵢⱼ+vᵢⱼ (22)

where the variables vᵢⱼ are independent and normally distributed random variables with zero mean and variance σᵢⱼ². If the measurement errors are considered as having a constant coefficient of variation (CV):

σᵢⱼ=CVxᵢⱼ (23)

It is possible to re-write (22) as:

yᵢ=Lᵢxᵢ+vᵢ (24)

where:

yᵢ=[yᵢ₁, . . . ,yᵢₙ_{ᵢ}]^{T} (25)

vᵢ=[vᵢ₁, . . . ,vᵢₙ_{ᵢ}]^{T} (26)

vᵢ^{˜}N(0,Σᵢ) (27)

$$\underline{{\sum }_{i}} = [ \begin{matrix}{σ}_{i1}^{2} & 0 & ⋯ & 0 \\ 0 & {σ}_{i2}^{2} & ⋯ & 0 \\ ⋮ & ⋰ & ⋰ & ⋮ \\ 0 & ⋯ & 0 & {σ}_{ini}^{2}\end{matrix} ] \qquad ( 28 )$$

and Lᵢ is a suitable (nᵢ x m) matrix. Lᵢ is required because yᵢ contains the set of all the measurements taken from the i-th individual (ie. nᵢ values) whilst xᵢ contains the set of all the actual values of the concentration in the i-th individual at the times T when any individual has a sample taken (ie. m values). Therefore Lᵢ is chosen such that Lᵢxᵢ contains the actual values of the concentration in the i-th individual at the times when a measurement is taken from the i-th individual. It is therefore the same size as yᵢ and each value of Lᵢxᵢ is the actual value of the concentration in the i-th individual at the time when the measurement for the value of yᵢ in the corresponding position was made.

By replacing xᵢ according to (18), (24), and hence (22), is rewritten as:

yᵢ=LᵢP⁻¹wᵢ+vᵢ (29)

FIG. 1 is a representation of the stochastic population model. As shown, standard prior distributions are chosen for the hyper-parameters \overline{λ}² and λ². In the case of FIG. 1 the distributions are gamma distributions. (In a gamma distribution Γ(a, b), the mean is a/b and the variance is a/b².)

The aim of the estimation procedure is to derive the joint (and then the marginal) probability distributions of \overline{λ}², λ², \overline{w}, w₁, . . . , w_{N} given the data. From the joint distribution it is possible to compute probability distributions of concentration curves and AUCs (both for the population and the individuals). Point estimates and their reliability can easily be derived.

Since the computation of the joint distribution cannot be derived in closed form, a Markov Chain Monte Carlo sampling scheme is used. In this case Gibbs Sampling is chosen, and then the samples are extracted, iteratively, from the following distributions:

$$\begin{matrix}p ( \frac{1}{{\overline{λ}}^{2}} \vert \overline{\underline{w}} ) = Γ ( \frac{m}{2} + {γ}_{1} , \frac{1}{2} {\overline{\underline{w}}}^{T} \overline{\underline{w}} + {γ}_{2} ) \\ p ( \frac{1}{{λ}^{2}} \vert \overline{\underline{w}} , {\underline{w}}_{1} , … , {\underline{w} }_{N} ) = Γ ( \frac{N}{2} + {γ}_{3} , \frac{1}{2} {\sum }_{i = 1}^{N} {( {\underline{w}}_{i} - \overline{\underline{w}} )}^{T} ( {\underline{w}}_{i} - \overline{\underline{w}} ) + {γ}_{4} )\end{matrix}$$

p(\overline{w}|\overline{λ}²,λ²,w₁, . . . ,w_{N})=N(A⁻¹B,A⁻¹)

p(wᵢ|λ²\overline{w},yᵢ)=N(C⁻¹D,C⁻¹)

where:

A=(\overline{λ}²+N*λ⁻²)I

$$\underline{B} = {\sum }_{i = 1}^{N} {λ}^{- 2} {\underline{\overline{w}}}_{i}$$

C=Lᵢ^{T}Σᵢ⁻¹Lᵢ+λ⁻²I

D=Lᵢ^{T}Σᵢ⁻¹yᵢ+λ⁻²\overline{w}

The population AUC thus determined provides a measure of the average exposure to a drug. It is further possible to determine the distribution of the individual AUCs within the population.

The method according to the present invention has been evaluated by comparing it to the Bailer and Yeh methods. The information relating to the comparison of the methods is shown in the tables below. Table 1 summarises the “experiments” which were performed. The analysed data set included plasma concentration data after a xenobiotic administration in 27 human subjects, each sampled at nine times (Rocchetti & Poggesi 1997).

In experiment 1 the methodology of the present invention was applied to the complete data set. After 1500 runs of the Gibbs Sampler (convergence is verified by using the Raftery Criterion; parameters of the prior distribution are fixed in the following way: γ₁=0.6, γ₂=3, γ₃=0.6, γ₄=10 CV is fixed to 0.1) the mean of the population AUC posterior distribution was found to be 805 and the standard error was found to be 104.

Experiments 2 and 3 were carried out to study the performance of the methodology of the present invention when the number of subjects involved in the study is reduced. In each experiment 100 randomly generated “synthetic” experiments were simulated by using a proportion (randomly selected) of the complete data set of experiment 1. A comparison of the results of experiments 2 and 3 with the results of experiment 1 is shown in table 2. It can be seen that although the mean of the population AUC posterior distribution means remains close the value obtained from the complete data set, the variance of the means increases markedly.

Experiment 4 was performed to compare methodology of the present invention with that of the Bailer method. Table 3 compares the results of the methodology of the invention, using the sampling scheme of experiment 4 (as given in Table 1), and the results of using the Bailer method, using the sampling scheme of experiment 4, with the results from experiment 1, in which the complete data set was used. The final column of the table is the difference in the results obtained from the two different methods. Again 100 “synthetic” experiments were conducted in which the samples were randomly drawn from the complete data set. It can be seen that two methods obtain almost exactly the same result if you consider the point estimate. However it can also be seen that the Bailer method significantly underestimates the Standard Error.

Experiments 5 and 6 were carried out to perform similar comparisons of results of the methodology of the present invention with the Yeh method when using the same sampling scheme. Again 100 “synthetic” experiments were performed. The results are shown in Tables 4 and 5 respectively. It can be seen from the results of this experiment that the Yeh method, as with the Bailer method, significantly underestimates the Standard Error.

Experiment 7 was performed to test the methodology of the present invention on a scheme that cannot be managed by the Bailer or Yeh methods (set out in Table 1). The scheme provides for sampling subjects in a completely “free” manner, in which each of the subjects can be sampled at any time. Again 100 “synthetic” experiments were performed by selecting random portions of the complete data set provided by experiment 1. Table 6 shows the mean and standard deviations of the mean and standard error of the population AUC posterior distribution derived from each of the “synthetic” experiments. In this case the estimates obtained are only slightly worse than the estimates obtained from the complete data set, however there is a big loss in terms of precision.

Whilst we have described above a specific embodiment of the invention, it will be appreciated that the invention may be practised otherwise than described. The description is not intended to limit the invention.

TABLE 1
Simulated Sampling Protocols

| | | Samples per | | |
|---|---|---|---|---|
| Experiment | Subjects | subject | Samples | Note |
| 1 | 27 | 9 | 243 | Complete |
| | | | | Data Set |
| 2 | 18 | 9 | 162 | |
| 3 | 9 | 9 | 81 | |
| 4 | 27 | 1 | 27 | Bailer |
| 5 | 9 | 3 | 27 | Yeh 1 |
| 6 | 9 | 3 | 27 | Yeh 2 |
| 7 | 10 | — | 27 | Free |

Simulated sampling protocols:
Yeh 1: subjects in session 1 are sampled at time points [t₁, t₄, t₇], those of session 2 are sampled at [t₂, t₅, t₈], and those of session 3 at [t₃, t₆, t₉];
Yeh 2: subjects in session 1 are sampled at time points [t₁, t₃, t₅], those of session 2 are sampled at [t₂, t₄, t₆], and those of session 3 at [t₇, t₈, t₉];
Free: one subject is fully sampled, one at time points [t₁, t₃, t₅, t₉], one at [t₂, t₃], one at [t₄, t₆], one at [t₅, t₉], one at [t₆], one at [t₇], one at [t₈], one at [t₇, t₈, t₉], and the last one at [t₂, t₁].

TABLE 2
Population AUC Estimates Using the Method of the Present Invention

| | Experiment | | |
|---|---|---|---|
| | 1 | 2 | 3 |
| Mean of Population AUC Posterior | 805 | 806 | 801 |
| Distribution Means | | | |
| Standard Deviation of Population AUC | N/A | 30 | 71 |
| Posterior Distribution Means | | | |
| Mean of Standard Errors | 104 | 127 | 179 |
| Standard Deviation of Standard Errors | N/A | 15 | 44 |

TABLE 3
Comparison of Bailer Method and the Method of the Present Invention

| | Exp | Present | Bailer | |
|---|---|---|---|---|
| | 1 | Invention | Method | Difference |
| Mean of Population AUC | 805 | 825 | 825 | 0.057 |
| Posterior Distribution Means | | | | |
| Standard Deviation of | | 49 | 48 | 19 |
| Population AUC Posterior | | | | |
| Distribution Means | | | | |
| Mean of Standard Errors | 104 | 123 | 70 | 53 |
| Standard Deviation of Standard | | 31 | 22 | 34 |
| Errors | | | | |

TABLE 4
Comparison of Yeh Method and the Method of the Present Invention
from Experiment 5

| | Exp | Present | Yeh | |
|---|---|---|---|---|
| | 1 | Invention | Method | Difference |
| Mean of Population AUC | 805 | 786 | 808 | −22 |
| Posterior Distribution Means | | | | |
| Standard Deviation of | | 72 | 76 | 20 |
| Population AUC Posterior | | | | |
| Distribution Means | | | | |
| Mean of Standard Errors | 104 | 149 | 79 | 69 |
| Standard Deviation of Standard | | 56 | 29 | 46 |
| Errors | | | | |

TABLE 5
Comparison of Yeh Method and the Method of the Present Invention
from Experiment 6

| | Exp | Present | Yeh | |
|---|---|---|---|---|
| | 1 | Invention | Method | Difference |
| Mean of Population AUC | 805 | 844 | 825 | 19 |
| Posterior Distribution Means | | | | |
| Standard Deviation of | | 94 | 95 | 43 |
| Population AUC Posterior | | | | |
| Distribution Means | | | | |
| Mean of Standard Errors | 104 | 201 | 92 | 109 |
| Standard Deviation of Standard | | 71 | 50 | 87 |
| Errors | | | | |

TABLE 6
Population AUC Estimates Using the Method of the Present Invention
on the “Free” Sampling Scheme of Experiment 7

Experiment

| | 1 | 2 |
|---|---|---|
| Mean of Population | 805 | 796 |
| AUC Posterior | | |
| Distribution Means | | |
| Standard Deviation of | | 87 |
| Population AUC | | |
| Posterior Distribution | | |
| Means | | |
| Mean of Standard Errors | 104 | 176 |
| Standard Deviation of | | 59 |
| Standard Errors | | |

1. A method of estimating the average exposure within a population of subjects to a pharmacological substance, administered according to a given protocol, by estimating the area under the concentration curve (AUC), characterised in that the area under the population concentration curve is estimated by the steps of:
obtaining measurements of the drug concentrations in each of the subjects at any time during the study, independently from the time when measurements of the drug concentration level in the other subjects are being taken;
building a hierarchical stochastic model composed of the population and of the individual levels;
determining the posterior probability distribution of the average AUC from the sample data and hence the average population AUC.

2. A method of determining the exposure of an individual and its precision within the sample of individuals given a pharmacological substance administered according to a given protocol by determining the area under the concentration-time curve, characterised in that the area under the individual concentration-time curve is estimated by the steps of:
obtaining measurements of the drug concentrations in each of the subjects at any time during the study, independently from the time when measurements of the drug concentration level in the other subjects are being taken;
building a hierarchical stochastic model composed of the population and of the individual levels;
determining the posterior probability distribution of the individual AUC from the sample data.

3. A method according to claim 1 or 2, wherein the posterior probability distribution is determined by using a Markov Chain Monte Carlo algorithm.

4. A method according to claim 1, 2 or 3, wherein the hierarchical model is obtained by:
modelling the variation of the population concentration level by a random walk;
modelling the individual's concentration level as a random walk with an expected value equal to the population curve; and
modelling the measurement error of each sample as an additive error.

5. A method according to any one of claims 1 to 4, wherein the Markov Chain Monte Carlo algorithm used is the Gibbs Sampling method.

6. A method of determining the therapeutic and toxic effects related to the administration of a pharmacological substance comprising the steps of:
administering the pharmacological substance to a population of subjects according to a given protocol;
determining the average of a measure of the therapeutic or toxic effect of the pharmacological substance within the population;
comparing this to the average total exposure to the pharmacological substance within the population which is determined by a method according to any one of claims 1 to 5.

7. A method of determining a safe exposure level of a subject to a pharmacological substance comprised of the steps of:
administering the pharmacological substance to a plurality of populations of subjects according a plurality of different protocols;
determining the average of a measure of toxic effect of the pharmacological substance within each population of subjects;
comparing the average measures of the toxic effects to the average total exposure to the pharmacological substance within each population which is determined by a method according to any one of claims 1 to 5.

8. A method of determining a beneficial exposure level of a subject to a pharmacological substance comprised of the steps of:
administering the pharmacological substance to a plurality of populations of subjects according a plurality of different protocols;
determining the average of a measure of therapeutic effect of the pharmacological substance within each population of subjects;
comparing the average measures of the therapeutic effects to the average total exposure to the pharmacological substance within each population which is determined by a method according to any one of claims 1 to 5.

9. A method of determining a dose protocol of a pharmacological substance comprising at least one of the steps of:
determining the therapeutic and toxic effects related to the administration of the pharmacological substance according to claim 6;
determining a safe exposure level of a subject to the pharmacological substance according to claim 7; and
determining a beneficial exposure level of a subject to the pharmacological substance according to claim 8.

10. A method of treatment comprised of preparing at least one dose of a pharmacological substance according to a dose protocol determined according to claim 9; and
administering said dose to the subject.

11. A computer program comprised of computer code means for estimating the average exposure within a population of subjects to a pharmacological substance, administered according to a given protocol, by estimating the area under the concentration curve (AUC), the computer code means being comprised of means to:
record measurements of the drug concentrations in each of the subjects at any time during the study, independently from the time when measurements of the drug concentration level in the other subjects are being taken;
build a hierarchical stochastic model composed of the population and of the individual levels;
determine the posterior probability distribution of the average AUC from the sample data and hence the average population AUC.

12. A computer program comprised of computer code means for determining the exposure of an individual and its precision within the sample of individuals given a pharmacological substance administered according to a given protocol by determining the area under the concentration-time curve, the computer code means being comprised of means to:
record measurements of the drug concentrations in each of the subjects at any time during the study, independently from the time when measurements of the drug concentration level in the other subjects are being taken;
build a hierarchical stochastic model composed of the population and of the individual levels; and
determine the posterior probability distribution of the individual AUC from the sample data.

13. A computer program according to claim 11 or 12, wherein the computer code means to determine the posterior probability from the recorded sample data uses Markov Chain Monte Carlo algorithm.

14. A computer program according to claim 11, 12 or 13, wherein the computer code means obtains the hierarchical model, used to determine the posterior probability distribution from the recorded sample data, by:
modelling the variation of the population concentration level by a random walk;
modelling the individual's concentration level as a random walk with an expected value equal to the population curve; and
modelling the measurement error of each sample as an additive error.

15. A computer program according to any one of claims 11 to 14, wherein the computer code means to determine the posterior probability from the recorded sample data uses the Gibbs Sampling method.
